(19) United States
(12) Patent Application Publication
(10) Pub. No.: US 2026/0022524 A1
(43) Pub. Date: Jan. 22, 2026

(54) PET WASTE REMOVAL DEVICE

(71) Applicant: Cornelius Tavarres, Queensbury, NY (US)

(72) Inventor: Cornelius Tavarres, Queensbury, NY (US)

(21) Appl. No.: 18/775,712

(22) Filed: Jul. 17, 2024

(51) Int. Cl. E01H 1/00; A01G 20/40; A47L 7/00; A47L 9/00; A47L 9/14; A47L 9/32; A61L 2/18; A61L 2/22; A61L 2/26

(52) U.S. Cl. CPC E01H 1/006; A47L 7/0061; A47L 9/009; A47L 9/14; A47L 9/325; A61L 2/18; A61L 2/22; A61L 2/26; A01G 20/40; A61L 2202/15; A61L 2202/16; A61L 2202/17

(57) ABSTRACT

A pet waste removal device to remove pet waste from a surface, pet waste removal device including a main body including a plurality of wheels disposed at bottom corners of the main body to rotate such that the main body is propelled in a forward direction, a waste collecting aperture disposed at a front portion of the main body to receive the pet waste therethrough, a reverse axle including a plurality of rakes disposed on a surface of the reverse axle, wherein the reverse axle rotates in a direction opposite to the rotation of the plurality of wheels to allow the plurality of rakes to pick up the pet waste and introduce the pet waste through the waste collecting aperture, and a sanitizer compartment to store sanitizer therein and nozzles to spray the sanitizer out from the sanitizer compartment.

### BACKGROUND

### 1. Field

The present general inventive concept relates generally to a waste removal device, and particularly, to a pet waste removal device.

### 2. Description of the Related Art

Removing pet waste from yards is a crucial part of lawn care considering decaying waste can contribute to the spread of harmful bacteria and pathogens.

Conventional methods of pet waste removal, however, can be incredibly time-consuming and labor-intensive.

Therefore, there is a need for a device that aids in removing pet waste, while also sanitizing the area where the pet waster used to be located.

### SUMMARY

The present general inventive concept provides a pet waste removal device.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a pet waste removal device to remove pet waste from a surface, pet waste removal device including a handlebar assembly to allow a user to control the pet waste removal device, a main body connected to the handlebar assembly, the main body including a plurality of wheels disposed at bottom corners of the main body to rotate such that the main body is propelled in a forward direction, a waste collecting aperture disposed at a front portion of the main body to receive the pet waste therethrough, a reverse axle including a plurality of rakes disposed on a surface of the reverse axle, wherein the reverse axle rotates in a direction opposite to the rotation of the plurality of wheels to allow the plurality of rakes to pick up the pet waste and introduce the pet waste through the waste collecting aperture, a mesh waste catcher disposed within the main body to receive the pet waste thereupon, and an internal hose to suck the pet waste up from the mesh waste catcher, a vacuum bag connected to the main body to receive the pet waste therein, such that the internal hose directs the pet waste from the mesh waste catcher into the vacuum bag, and a sanitizer compartment to store sanitizer therein, the sanitizer compartment including a plurality of sanitizer compartment spray nozzles disposed at a bottom surface of the sanitizer compartment to spray the sanitizer out from the sanitizer compartment.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a front-top-right-angled view of a pet waste removal device, according to an exemplary embodiment of the present general inventive concept; and

FIG. 2 illustrates a front-top-right-angled view of a pet waste removal device, according to another exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Pet Waste Removal Device 100
Handlebar Assembly 110
Handlebar 111
Handlebar Arms 112
Handlebar Arm Adjustment Knobs 112a
Steering Extensions 113
Throttle 114
Brake 115
Sanitizer Spray Lever 116
Sanitizer Spray Cable 117
Vacuum Hose Bracket 118
Phone Charging Holder 119
Main Body 120
Power Button 121
LED 122
Battery Compartment 123
Battery 123a
Charging Port 123b
Internal Hose 124
Sensors 125
Waste Collecting Aperture 126
Mesh Waste Catcher 126a
Reverse Axle 127
Rakes 127a
Wheels 128
Main Body Height Adjustment Knob 129
Vacuum Bag 130
Handheld Vacuum Assembly 140
Vacuum Hose 141
Vacuum Head 142
Vacuum Head Sprinkler 143
Handheld Vacuum Switch 144
Sanitizer Compartment 150
Sanitizer Compartment Cap 151
Sanitizer Compartment Spray Nozzles 152
Sanitizer 153
Control Panel Display Screen 160
Motor 170
Suspension Platform 180
Pet Waste Removal Device 200
Handlebar Assembly 210
Handlebar 211
Handlebar Arms 212
Handlebar Arm Adjustment Knobs 212a
Steering Extensions 213
Throttle 214
Brake 215
Sanitizer Spray Lever 216
Sanitizer Spray Cable 217
Vacuum Hose Bracket 218
Phone Charging Holder 219
Main Body 220
Power Button 221
LED 222
Battery Compartment 223
Battery 223a
Charging Port 223b
Internal Hose 224
Sensors 225
Waste Collecting Aperture 226
Mesh Waste Catcher 226a
Reverse Axle 227
Brushes 227a
Wheels 228
Main Body Height Adjustment Knob 229
Vacuum Bag 230
Handheld Vacuum Assembly 240
Vacuum Hose 241
Vacuum Head 242
Vacuum Head Sprinkler 243
Handheld Vacuum Switch 244
Sanitizer Compartment 250
Sanitizer Compartment Cap 251
Sanitizer Compartment Spray Nozzles 252
Sanitizer 253
Control Panel Display Screen 260
Motor 270
Suspension Platform 280

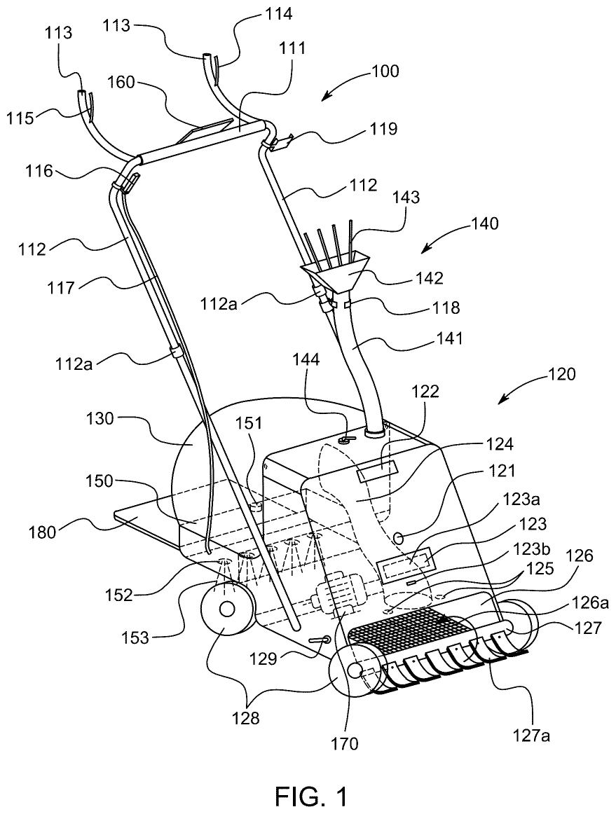

FIG. 1 illustrates a front-top-right-angled view of a pet waste removal device 100, according to an exemplary embodiment of the present general inventive concept.

The pet waste removal device 100, and all components therein and/or connected thereto, may be constructed from at least one of metal, plastic, stone, ceramics, wood, silicone, paper, cloth, circuitry, leather, circuitry, wiring, liquids, and rubber, etc., but is not limited thereto, and can be constructed from any material known to one of ordinary skill in the art.

Referring to FIG. 1, the pet waste removal device 100 may include a handlebar assembly 110, a main body 120, a vacuum bag 130, a handheld vacuum assembly 140, a sanitizer compartment 150, a control panel display screen 160, a motor 180, and a suspension platform 180, but is not limited thereto.

The handlebar assembly 110 may include a handlebar 111, a plurality of handlebar arms 112, a plurality of handlebar arm adjustment knobs 112a, a plurality of steering extensions 113, a throttle 114, a brake 115, a sanitizer spray lever 116, a sanitizer spray cable 117, a vacuum hose bracket 118, and a phone charging holder 119, but is not limited thereto.

The main body 120 may include a power button 121, an LED (light emitting diode) 122, a battery compartment 123, a battery 123a, a charging port 123b, an internal hose 124, a plurality of sensors 125, a waste collecting aperture 126, a mesh waste catcher 126a, a reverse axle 127, a plurality of rakes 127a, a plurality of wheels 128, and a main body height adjustment knob 129, but is not limited thereto.

The handlebar 111 is a portion of the pet waste removal device 100 that may be grasped by a user to allow the user to pull, push, and manually control the pet waste removal device 100. The handlebar 111 may be connected to the main body 120 via the plurality of handlebar arms 112, which each extend downward and diagonally from end portions of the handlebar 111. A length of the plurality of handlebar arms 112 may be adjusted by twisting the plurality of handlebar arm adjustment knobs 112a, such that the plurality of handlebar arms 112 can be extended to be longer when the plurality of handlebar arm adjustment knobs 112a are twisted in a first direction, for example, and such that the plurality of handlebar arms 112 can be retracted to be shorter when the plurality of handlebar arm adjustment knobs 112a are twisted in a second direction, for example. Alternatively, twisting the plurality of handlebar arm adjustment knobs 112a may loosen two portions of each of the plurality of handlebar arms 112, such that they can move independently with respect to each other to lengthen or shorten each of the plurality of handlebar arms 112.

The plurality of steering extensions 113 may be disposed at opposite ends of the handlebar 111, and may allow for easier steering of the pet waste removal device 100.

The throttle 114 may be disposed at a first one of the plurality of steering extensions 113 to allow the pet waste removal device 100 to move forward automatically when the throttle 114 is squeezed. As such, the throttle 114 may activate the motor 170, thus providing power to the wheels 128, causing them to spin.

The brake 115 may be disposed at a second one of the plurality of steering extensions 113 to allow the pet waste removal device 100 to stop when the brake 115 is squeezed.

The sanitizer spray lever 116 may be disposed on and/or connected to at least one of the handlebar 111, the plurality of handlebar arms 112, and the plurality of steering extensions 113, and may be connected to the sanitizer spray compartment 150 via the sanitizer spray cable 117.

The vacuum hose bracket 118 and the phone charging holder 119 may be disposed on and/or connected to at least one of the handlebar 111, the plurality of handlebar arms 112, and the plurality of steering extensions 113, but is not limited thereto. The phone charging holder 119 may be designed to hold and charge a mobile device such as a cell phone.

The vacuum bag 130 may be removably and replaceably connected to the main body 120 as well as the handheld vacuum assembly 140, such that any waste that is picked up by the pet waste removal device 100 may be directed to the vacuum bag 130.

The handheld vacuum assembly 140 may include a vacuum hose 141, a vacuum head 142, a vacuum head sprinkler 143, and a handheld vacuum switch 144, but is not limited thereto.

The vacuum hose bracket 118 may be designed to hold the vacuum hose 141 when the handheld vacuum assembly 140 is not in use.

The vacuum hose 141 may be connected to the vacuum head 142 at a first end thereof, and may be connected to the vacuum bag 130 at a second end thereof.

The handheld vacuum switch 144 may be disposed on the main body 120, but is not limited thereto, and may act to switch the sucking from the internal hose 124 to the vacuum hose 141, or alternatively, may simply power on the handheld vacuum assembly 140.

When the handheld vacuum switch 144 is turned on, the motor 170 may operate to allow a suction to be generated by the vacuum head 142, in order to allow the user to use the handheld vacuum assembly 140 to vacuum pet waste and debris, such that the pet waste travels through the vacuum head 142, then through the vacuum hose 141, and then into the vacuum bag 130.

The sanitizer compartment 150 may include a sanitizer compartment cap 151, a plurality of sanitizer compartment spray nozzles 152, and a sanitizer 153, but is not limited thereto.

The vacuum head sprinkler 143 may include a plurality of nozzles that spray the sanitizer 150 based on a user's preference, such that the spray of the sanitizer 153 may be controlled via the control panel display screen 160.

The sanitizer compartment 150 may be connected to the main body 120 and may house thereinside the sanitizer 153, which may be any type of liquid sanitizer known to one of ordinary skill in the art.

The sanitizer 153 may be introduced into the sanitizer compartment 150 by removing the sanitizer compartment cap 151, thus allowing access into the sanitizer compartment 150.

The user may squeeze the sanitizer spray lever 116 to cause the sanitizer 153 to be expelled downward out from the sanitizer compartment spray nozzles 152. Alternatively, the user may control the sanitizer compartment spray nozzles 152 to spray the sanitizer 153 out therefrom via the control panel display screen 160.

The battery compartment 123 may house the battery 123a thereinside, and the charging port 123b may allow a user to connect an external cord thereto to recharge the battery 123a.

The internal hose 124 may be connected to the vacuum bag 130 at a first end and may have a second end disposed over the mesh waste catcher 126a.

The mesh waste catcher 126a is herein depicted as a mesh surface to allow for any liquid to drain therethough. However, the mesh waste catcher 126a is not limited to a mesh surface, and may alternatively be a non-mesh surface.

Thus, when the power button 121 is depressed (thus turning on/powering on the pet waste removal device 100), the an LED 122 may indicate that the pet waste removal device 100 is powered on, and the motor 170 may (1) cause the internal hose 124 to begin suction, and (2) cause the reverse axle 127 to turn in a backward direction (i.e., opposite with respect to a forward moving direction of the plurality of wheels 128).

The reverse axle 127 may have the plurality of rakes 127a attached thereto to allow pet waste to be picked up and placed into the main body 120.

When the pet waste removal device 100 is powered on via the power button 121 and the user squeezes the throttle 114, the plurality of wheels 128 may being to turn such that the pet waste removal device 100, while the reverse axle 127 turns in a backward direction to allow the plurality of rakes 127a to pick up any pet waste and throw the pet waste through the waste collecting aperture 126 such that the pet waste is collected onto the mesh waste catcher 126a.

In other to prevent the internal hose 124 from being powered on the entire time after the power button 121 is depressed (and thus conserving batter life), when the plurality of sensors 125 detect that there is pet waste disposed on the mesh waste catcher 126a, the internal hose 124 may then begin suction such that the pet waste disposed on the mesh waste catcher 126a is sucked up through the internal hose 124 and guided therethrough and into the vacuum bag 130.

The main body height adjustment knob 129 may function to allow the main body 120 to be raised or lowered with respect to the plurality of wheels 128.

The control panel display screen 160 may include a touch screen (or may have pressable buttons disposed thereupon) that allows the user to select between various modes of the pet waste removal device 100, including, but not limited to, a mode where the internal hose 124 is sucking, a mode where the vacuum hose 141 is sucking, a mode where the plurality of wheels 128 are turning, a mode to allow the vacuum head sprinkler 143 to spray the sanitizer 153, or any other analog operation of the pet waste removal device 100.

The motor 170 may provide the mechanical aspects to activate the sucking, the sanitizer spraying, component-movement, and propulsion of the various components within the pet waste removal device 200.

The control panel display screen 160 may include an internal CPU.

The CPU may include electronic circuitry to carry out instructions of a computer program by performing basic arithmetic, logical, control and input/output (I/O) operations specified by the instructions. The CPU may include an arithmetic logic unit (ALU) that performs arithmetic and logic operations, processor registers that supply operands to the ALU and store the results of ALU operations, and a control unit that fetches instructions from memory and “executes” them by directing the coordinated operations of the ALU, registers and other components. The CPU may also include a microprocessor and a microcontroller. The transmitter CPU may be a local computer device, a remote server, or cloud computing device.

The CPU may also include a storage unit, a random access memory (RAM), a read-only memory (ROM), a hard disk, a flash drive, a database connected to the Internet, cloud-based storage, Internet-based storage, or any other type of storage unit.

The CPU may access the Internet via an internal communication unit to allow the CPU to access a website, and/or may allow for communication with a mobile application and/or a software application. For ease of description, the mobile and/or the software application will be hereinafter referred to as an app. The app may be downloaded from the Internet to be stored on the storage unit of the CPU.

The CPU may also have a global positioning service (GPS) device installed therein, to allow the CPU to be located via a GPS signal.

Thus, the CPU may allow the control panel display screen 160 to communicate with all of the components within the pet waste removal device 100.

The suspension platform 180 may be foldable, retractable, and/or removable, and may allow a user to stand thereupon so that the user may ride the pet waste removal device 100.

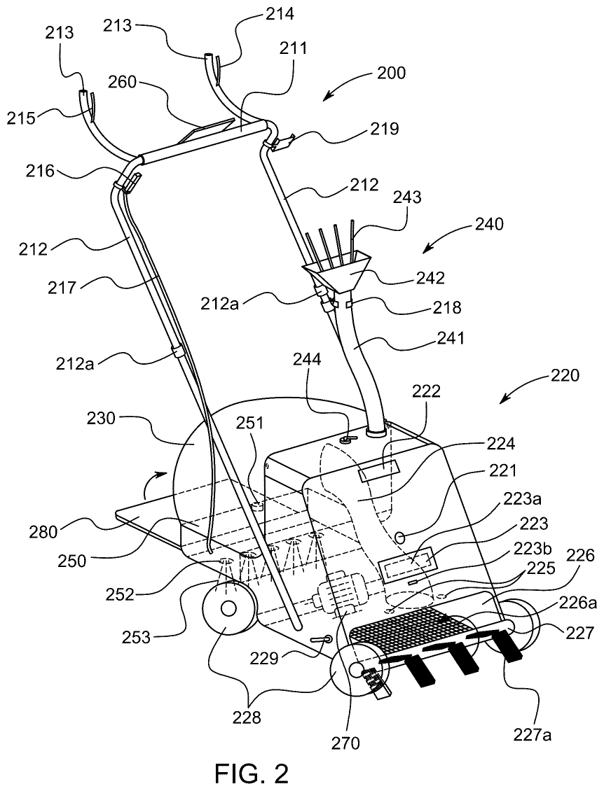

FIG. 2 illustrates a front-top-right-angled view of a pet waste removal device 200, according to another exemplary embodiment of the present general inventive concept.

The pet waste removal device 200, and all components therein and/or connected thereto, may be constructed from at least one of metal, plastic, stone, ceramics, wood, silicone, paper, cloth, circuitry, leather, circuitry, wiring, liquids, and rubber, etc., but is not limited thereto, and can be constructed from any material known to one of ordinary skill in the art.

Referring to FIG. 2, the pet waste removal device 200 may include a handlebar assembly 210, a main body 220, a vacuum bag 230, a handheld vacuum assembly 240, a sanitizer compartment 250, a control panel display screen 260, a motor 270, and a suspension platform 280, but is not limited thereto.

The handlebar assembly 210 may include a handlebar 211, a plurality of handlebar arms 212, a plurality of handlebar arm adjustment knobs 212a, a plurality of steering extensions 213, a throttle 214, a brake 215, a sanitizer spray lever 216, a sanitizer spray cable 217, a vacuum hose bracket 218, and a phone charging holder 219, but is not limited thereto.

The main body 220 may include a power button 221, an LED (light emitting diode) 222, a battery compartment 223, a battery 123a, a charging port 223b, an internal hose 224, a plurality of sensors 225, a waste collecting aperture 226, a mesh waste catcher 226a, a reverse axle 227, a plurality of brushes 227a, a plurality of wheels 228, and a main body height adjustment knob 229, but is not limited thereto.

The handlebar 211 is a portion of the pet waste removal device 200 that may be grasped by a user to allow the user to pull, push, and manually control the pet waste removal device 200. The handlebar 211 may be connected to the main body 220 via the plurality of handlebar arms 212, which each extend downward and diagonally from end portions of the handlebar 211. A length of the plurality of handlebar arms 212 may be adjusted by twisting the plurality of handlebar arm adjustment knobs 212a, such that the plurality of handlebar arms 212 can be extended to be longer when the plurality of handlebar arm adjustment knobs 212a are twisted in a first direction, for example, and such that the plurality of handlebar arms 212 can be retracted to be shorter when the plurality of handlebar arm adjustment knobs 212a are twisted in a second direction, for example. Alternatively, twisting the plurality of handlebar arm adjustment knobs 212a may loosen two portions of each of the plurality of handlebar arms 212, such that they can move independently with respect to each other to lengthen or shorten each of the plurality of handlebar arms 212.

The plurality of steering extensions 213 may be disposed at opposite ends of the handlebar 211, and may allow for easier steering of the pet waste removal device 200.

The throttle 214 may be disposed at a first one of the plurality of steering extensions 213 to allow the pet waste removal device 200 to move forward automatically when the throttle 214 is squeezed. As such, the throttle 214 may activate the motor 270, thus providing power to the wheels 228, causing them to spin.

The brake 215 may be disposed at a second one of the plurality of steering extensions 213 to allow the pet waste removal device 200 to stop when the brake 215 is squeezed.

The sanitizer spray lever 216 may be disposed on and/or connected to at least one of the handlebar 211, the plurality of handlebar arms 212, and the plurality of steering extensions 213, and may be connected to the sanitizer spray compartment 250 via the sanitizer spray cable 217.

The vacuum hose bracket 218 and the phone charging holder 219 may be disposed on and/or connected to at least one of the handlebar 211, the plurality of handlebar arms 212, and the plurality of steering extensions 213, but is not limited thereto. The phone charging holder 219 may be designed to hold and charge a mobile device such as a cell phone.

The vacuum bag 230 may be removably and replaceably connected to the main body 220 as well as the handheld vacuum assembly 240, such that any waste that is picked up by the pet waste removal device 200 may be directed to the vacuum bag 230.

The handheld vacuum assembly 240 may include a vacuum hose 241, a vacuum head 242, a vacuum head sprinkler 243, and a handheld vacuum switch 244, but is not limited thereto.

The vacuum hose bracket 218 may be designed to hold the vacuum hose 141 when the handheld vacuum assembly 240 is not in use.

The vacuum hose 241 may be connected to the vacuum head 242 at a first end thereof, and may be connected to the vacuum bag 230 at a second end thereof.

The handheld vacuum switch 244 may be disposed on the main body 220, but is not limited thereto, and may act to switch the sucking from the internal hose 224 to the vacuum hose 241, or alternatively, may simply power on the handheld vacuum assembly 240.

When the handheld vacuum switch 244 is turned on, the motor 270 may operate to allow a suction to be generated by the vacuum head 242, in order to allow the user to use the handheld vacuum assembly 240 to vacuum pet waste and debris, such that the pet waste travels through the vacuum head 242, then through the vacuum hose 241, and then into the vacuum bag 230.

The sanitizer compartment 250 may include a sanitizer compartment cap 251, a plurality of sanitizer compartment spray nozzles 252, and a sanitizer 253, but is not limited thereto.

The vacuum head sprinkler 243 may include a plurality of nozzles that spray the sanitizer 250 based on a user's preference, such that the spray of the sanitizer 253 may be controlled via the control panel display screen 260.

The sanitizer compartment 250 may be connected to the main body 220 and may house thereinside the sanitizer 253, which may be any type of liquid sanitizer known to one of ordinary skill in the art.

The sanitizer 153 may be introduced into the sanitizer compartment 250 by removing the sanitizer compartment cap 251, thus allowing access into the sanitizer compartment 250.

The user may squeeze the sanitizer spray lever 216 to cause the sanitizer 153 to be expelled downward out from the sanitizer compartment spray nozzles 252. Alternatively, the user may control the sanitizer compartment spray nozzles 252 to spray the sanitizer 253 out therefrom via the control panel display screen 260.

The battery compartment 223 may house the battery 223a thereinside, and the charging port 223b may allow a user to connect an external cord thereto to recharge the battery 223a.

The internal hose 224 may be connected to the vacuum bag 230 at a first end and may have a second end disposed over the mesh waste catcher 226a.

The mesh waste catcher 226a is herein depicted as a mesh surface to allow for any liquid to drain therethough. However, the mesh waste catcher 226a is not limited to a mesh surface, and may alternatively be a non-mesh surface.

Thus, when the power button 221 is depressed (thus turning on/powering on the pet waste removal device 200), the an LED 222 may indicate that the pet waste removal device 200 is powered on, and the motor 270 may (1) cause the internal hose 224 to begin suction, and (2) cause the reverse axle 227 to turn in a backward direction (i.e., opposite with respect to a forward moving direction of the plurality of wheels 228).

The reverse axle 227 may have the plurality of brushes 227a attached thereto to allow pet waste to be picked up and placed into the main body 220.

When the pet waste removal device 200 is powered on via the power button 121 and the user squeezes the throttle 214, the plurality of wheels 228 may being to turn such that the pet waste removal device 200, while the reverse axle 227 turns in a backward direction to allow the plurality of brushes 227a to pick up any pet waste and throw the pet waste through the waste collecting aperture 226 such that the pet waste is collected onto the mesh waste catcher 226a.

In other to prevent the internal hose 224 from being powered on the entire time after the power button 221 is depressed (and thus conserving batter life), when the plurality of sensors 225 detect that there is pet waste disposed on the mesh waste catcher 226a, the internal hose 224 may then begin suction such that the pet waste disposed on the mesh waste catcher 226a is sucked up through the internal hose 224 and guided therethrough and into the vacuum bag 230.

The main body height adjustment knob 229 may function to allow the main body 120 to be raised or lowered with respect to the plurality of wheels 128.

The control panel display screen 260 may include a touch screen (or may have pressable buttons disposed thereupon) that allows the user to select between various modes of the pet waste removal device 200, including, but not limited to, a mode where the internal hose 224 is sucking, a mode where the vacuum hose 241 is sucking, a mode where the plurality of wheels 228 are turning, a mode to allow the vacuum head sprinkler 243 to spray the sanitizer 253, or any other analog operation of the pet waste removal device 200.

The motor 270 may provide the mechanical aspects to activate the sucking and propulsion of the various components within the pet waste removal device 200.

The control panel display screen 260 may include an internal CPU.

The CPU may include electronic circuitry to carry out instructions of a computer program by performing basic arithmetic, logical, control and input/output (I/O) operations specified by the instructions. The CPU may include an arithmetic logic unit (ALU) that performs arithmetic and logic operations, processor registers that supply operands to the ALU and store the results of ALU operations, and a control unit that fetches instructions from memory and “executes” them by directing the coordinated operations of the ALU, registers and other components. The CPU may also include a microprocessor and a microcontroller. The transmitter CPU may be a local computer device, a remote server, or cloud computing device.

The CPU may also include a storage unit, a random access memory (RAM), a read-only memory (ROM), a hard disk, a flash drive, a database connected to the Internet, cloud-based storage, Internet-based storage, or any other type of storage unit.

The CPU may access the Internet via an internal communication unit to allow the CPU to access a website, and/or may allow for communication with a mobile application and/or a software application. For ease of description, the mobile and/or the software application will be hereinafter referred to as an app. The app may be downloaded from the Internet to be stored on the storage unit of the CPU.

The CPU may also have a global positioning service (GPS) device installed therein, to allow the CPU to be located via a GPS signal.

Thus, the CPU may allow the control panel display screen 260 to communicate with all of the components within the pet waste removal device 200.

The suspension platform 280 may be foldable, retractable, and/or removable, and may allow a user to stand thereupon so that the user may ride the pet waste removal device 200.

The present general inventive concept may include a pet waste removal device 100 to remove pet waste from a surface, pet waste removal device 100 including a handlebar assembly 110 to allow a user to control the pet waste removal device 100, a main body 120 connected to the handlebar assembly 110, the main body 120 including a plurality of wheels 128 disposed at bottom corners of the main body 120 to rotate such that the main body 120 is propelled in a forward direction, a waste collecting aperture 126 disposed at a front portion of the main body 120 to receive the pet waste therethrough, a reverse axle 127 including a plurality of rakes 127a disposed on a surface of the reverse axle 127, wherein the reverse axle 127 rotates in a direction opposite to the rotation of the plurality of wheels 128 to allow the plurality of rakes 127a to pick up the pet waste and introduce the pet waste through the waste collecting aperture 126, a mesh waste catcher 126a disposed within the main body 120 to receive the pet waste thereupon, and an internal hose 124 to suck the pet waste up from the mesh waste catcher, a vacuum bag 130 connected to the main body 120 to receive the pet waste therein, such that the internal hose 124 directs the pet waste from the mesh waste catcher 126a into the vacuum bag 130, and a sanitizer compartment 150 to store sanitizer 153 therein, the sanitizer compartment 150 including a plurality of sanitizer compartment spray nozzles 152 disposed at a bottom surface of the sanitizer compartment 150 to spray the sanitizer 153 out from the sanitizer compartment 150.

A main purpose of the present general inventive concept is to provide users with a pet waste removal tool that streamlines waste management while protecting families and pets from harmful diseases. Ingenious and practical, the present general inventive concept offers a modern lightweight product that streamlines the pet waste removal process for private or commercial backyards, lawns, or parks. The present general inventive concept introduces a novel steel, aluminum, or plastic upright fan rake powered by either manual force, battery, gas, or electric power. With various rake styles available, including a steel grid with metal rakes facing diagonally toward the ground, the present general inventive concept offers versatility and efficiency in cleaning up dog waste from yards of all sizes and surfaces. With the addition of a sanitizer function, the battery-operated dual nozzle sprayer or manual distribution system allows users to sanitize areas where pet waste was removed, effectively eliminating germs and bacteria from the direct site. For areas with rocks or mulch where rakes may not be effective, this device employs a retractable vacuum hose attached to the side, permitting users to easily collect dog waste from hard-to-reach areas, ensuring thorough cleaning and sanitation.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A pet waste removal device to remove pet waste from a surface, pet waste removal device comprising:
a handlebar assembly to allow a user to control the pet waste removal device;
a main body connected to the handlebar assembly, the main body comprising:
a plurality of wheels disposed at bottom corners of the main body to rotate such that the main body is propelled in a forward direction,
a waste collecting aperture disposed at a front portion of the main body to receive the pet waste therethrough,
a reverse axle including a plurality of rakes disposed on a surface of the reverse axle, wherein the reverse axle rotates in a direction opposite to the rotation of the plurality of wheels to allow the plurality of rakes to pick up the pet waste and introduce the pet waste through the waste collecting aperture,
a mesh waste catcher disposed within the main body to receive the pet waste thereupon, and
an internal hose to suck the pet waste up from the mesh waste catcher;
a vacuum bag connected to the main body to receive the pet waste therein, such that the internal hose directs the pet waste from the mesh waste catcher into the vacuum bag; and
a sanitizer compartment to store sanitizer therein, the sanitizer compartment comprising:
a plurality of sanitizer compartment spray nozzles disposed at a bottom surface of the sanitizer compartment to spray the sanitizer out from the sanitizer compartment.
